SEQUENCE LISTING (6 sequences)

SEQ ID NO: 1 - DNA, 72 bp, synthetic construct
  source 1..72: mol_type = other DNA
ggcaggaagacaaacaggtcgtagtggaaactgtccaccgtagaccggttatctagtggtctgtggtgctgt

SEQ ID NO: 2 - DNA, 33 bp, synthetic construct
  source 1..33: mol_type = other DNA
caccgtagaccggttatctagtggtctgtggtg

SEQ ID NO: 3 - DNA, 46 bp, synthetic construct
  source 1..46: mol_type = other DNA
acacgatgggggggtatgatttgatgtggttgttgcatgatcgtgg

SEQ ID NO: 4 - DNA, 44 bp, synthetic construct
  source 1..44: mol_type = other DNA
gcctgtaaggtggtcggtgtggcgagtgtgttaggagagattgc

SEQ ID NO: 5 - DNA, 45 bp, synthetic construct
  source 1..45: mol_type = other DNA
ttttttggcgattcgtgatctctgctctcggtttcgcgttcgttc

SEQ ID NO: 6 - DNA, 42 bp, synthetic construct
  source 1..42: mol_type = other DNA
ttttttcgtgcagtacgccaacctttctcatgcgctgcccct

(19) United States
(12) Patent Application Publication
(10) Pub. No.: US 2026/0262973 A1
(43) Pub. Date: Sep. 10, 2026

(54) Optical Aptamer Biosensors for In-Vivo Sensing

(71) Applicant: University of Cincinnati, Cincinnati, OH (US)

(72) Inventor: Jason Heikenfeld, Cincinnati, OH (US)

(21) Appl. No.: 19/163,544

(22) PCT Filed: Mar. 8, 2024
(86) PCT No.: PCT/US2024/019276

§ 371 Date: Sep. 9, 2025

Related U.S. Application Data
(60) Provisional application No. 63/451,011, filed on Mar. 9, 2023.
(60) Provisional application No. 63/452,557, filed on Mar. 16, 2023.
(60) Provisional application No. 63/456,610, filed on Apr. 3, 2023.
(60) Provisional application No. 63/538,313, filed on Sep. 14, 2023.

(51) Int. Cl. A61B 5/1473; A61B 5/00; A61B 5/145; A61B 5/1459

(52) U.S. Cl. CPC A61B 5/14735; A61B 5/14514; A61B 5/1459; A61B 5/685; A61B 2562/0233

(57) ABSTRACT

A device for continually sensing at least one analyte in a sample fluid via measurement of the analyte is provided. The device includes an optically transparent component adapted for in-vivo placement. The device also includes a plurality of aptamers bound to the optically transparent component. The aptamers are capable of binding to the analyte. Also, the aptamers carry at least one tag that changes in at least one optically measurable property when the aptamers bind to the analyte. Additionally, an optical source and detector coupled to the optically transparent component can measure the optically measurable property of the aptamers.

### CROSS REFERENCE TO RELATED APPLICATIONS

This application claims priority to U.S. Provisional Application Ser. No. 63/451,011, filed Mar. 9, 2023, U.S. Provisional Application Ser. No. 63/452,557, filed Mar. 16, 2023, U.S. Provisional Application Ser. No. 63/456,610, filed Apr. 3, 2023, and U.S. Provisional Application Ser. No. 63/538,313, filed Sep. 14, 2023, which applications are hereby incorporated by reference in their entirety.

### FIELD OF THE INVENTION

This invention relates generally to construction and placement of optical aptamer biosensors for in-vivo use.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an target analyte of interest via the use of an aptamer sequence that specifically binds to the analyte of interest. These sensors include aptamers attached to an electrode or an optical surface, wherein each of the aptamers has a redox active molecule (redox tag) or a fluorescent tag and/or quencher tag attached thereto. These types of sensors can be referred to as electrochemical and as optical aptamer sensors. When an analyte binds to the aptamer, the aptamer changes shape, moving the tag such that a measurable electrical or optical signal is changed. When used in this manner, then, aptamers are an example of an affinity-based biosensor.

A major unresolved challenge for aptamer sensors and other affinity-based biosensors (particularly those where the aptamers are bonded to the working electrode) is the ability to place the sensors in the in-vivo testing environment. The patent literature and academic publications are dominated by aptamer biosensor devices based on in-vitro (bench-top) testing or based on invasive surgical implantation of a wired aptamer biosensor which is hardly practical for real world use. Just recently, electrochemical aptamer biosensors breakthroughs in longevity and early human testing have occurred for electrochemical aptamer sensors. However, optical aptamer sensors remain significantly underdeveloped compared to electrochemical aptamers, despite the fact that aptamer biosensors began with optical detection (molecular beacon technology) and despite the fact that academic in-vitro demonstrations have been around for more than a decade. There are numerous challenges that must be solved before moving optical aptamer biosensing into the body. These challenges include but are not limited to:
1) the ability to measure for at least 3 days in-vivo and ideally >7 days;
2) the ability to measure analytes >3 kDa in size, which precludes tight membrane protection;
3) a need for more manufacturable approaches than artisanal laboratory methods;
4) a need to increase optical signal such that optical measurement components can be small in form factor, low cost and accurate, or to reduce intensity or duration of optical excitation which can photobleach dyes over time;
5) the ability to multiplex without cross-talk between analytes being measured;
6) safe and ergonomic insertion and removal from the body;
7) reliable operation in the body such that, abrasion or pressure does not affect sensor signal;
8) shelf stability and sterility;
9) methods to minimize the need for repeated or ideally any calibration at all;
10) methods to achieve detection ranges less than 1 nM; and
11) other challenges that must be addressed for an economically and clinically impactful device.

Novel approaches for optical aptamer sensors are needed which reduce or eliminate these challenges such the sensors are more broadly attractive for in-vivo biosensing.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for continually sensing at least one analyte in a sample fluid via measurement of the analyte. The device includes at least one optically transparent component and a plurality of aptamers bound to the optically transparent component where the aptamers bind to the analyte and carry at least one optical tag that changes in at least one optically measurable property when the aptamers bind to the analyte. Further, the device includes at least one optical source and detector coupled to the optically transparent component capable of measuring the measurable optical property of the aptamers.

The present invention involves a device for continually sensing at least one analyte in a sample fluid via measurement of the analyte. The device includes at least one optically transparent component adapted for in-vivo placement. The device also includes a plurality of aptamers bound to the optically transparent component. The aptamers are capable of binding to the analyte. Also, the aptamers carry at least one tag that changes in at least one optically measurable property when the aptamers bind to the analyte. Additionally, at least one optical source and detector coupled to the optically transparent component is capable of measuring the optically measurable property of the aptamers.

In one embodiment, the aptamers comprise at least one fluorescent tag, and at least one optical quencher tag. In another embodiment, the aptamers comprise at least one first fluorescent tag, and at least one second fluorescent tag, wherein energy is transferred from the first fluorescent tag to the second fluorescent tag. In one embodiment, the optically transparent component is porous and carries aptamers internally. In another embodiment, the optically transparent component is coated with a porous material that carries aptamers.

In one embodiment, the porous material has pores that have an average size selected from the group consisting of greater than 3, 10, and 30 nm and a width selected from the group consisting of less than 50, 100, 200, and 500 nm. In another embodiment, the optically transparent material is a waveguide. In one embodiment, the optically transparent component is a hydrogel. In another embodiment, the optically transparent component is porous silica. In one embodiment, the plurality of aptamers includes at least a first subset of aptamers that bind to a first analyte and a second subset of aptamers that bind to a second analyte.

In another embodiment, the optically transparent material is a plurality of waveguides. In one embodiment, at least 90% of the aptamers will remain attached for at least 3 days when tested for 3 days of operation in serum at 37 degrees Celsius. In another embodiment, the porous material has a porous surface area of at least 20 m²/g. In one embodiment, the optically transparent component has a surface area to volume that is greater than 40,0000 by an amount selected from the group consisting of 2×, 10×, 50×, 200×, and 1000× greater than the equivalent surface area to volume of a smooth planar 100 μm diameter waveguide.

In another embodiment, the optically transparent component is a bundle of optical fibers with at least 1 micrometer of separation between the optical fibers. In one embodiment, the optically transparent component is non-cylindrical. In another embodiment, the aptamers comprise a plurality of fluorescent tags that are separated by at least 5 nm. In one embodiment, the optically transparent component has a surface and the surface comprises the aptamers and an antifouling chemistry. In another embodiment, the antifouling chemistry has a mixed charge at the interface with the sample fluid. In one embodiment, the antifouling chemistry has zwitterionic charge. In another embodiment, the antifouling chemistry has a charge. In one embodiment, the antifouling chemistry has a net charge and the charge is negative. In another embodiment, the charge is less than 50% of an equivalent of a net charge of 1 charge per nm².

In one embodiment, the antifouling chemistry has a plurality of charged molecular brushes interspersed in between the aptamers. In another embodiment, the molecular brushes are >2 nm in length. In one embodiment, the device also includes a protective layer that protects the surface from fouling in between the aptamers. The protective layer comprises a monolayer of mixed charge molecules that form a boundary with the sample fluid. The boundary layer and the mixed charge monolayer having a Debye length. Also, the molecular brush molecules have a net charge near the aptamers which extend the Debye length from the boundary layer with the mixed charge monolayer by at least twice the length.

In another embodiment, the device also includes at least one membrane between the optically transparent component and the sample fluid. In one embodiment, the device also includes at least one mechanically protective element between the optically transparent component and the sample fluid. In another embodiment, the mechanically protective element is porous to the analyte. In one embodiment, the optically transparent component is formed on at least one metal wire. In another embodiment, the metal wire is tantalum.

In one embodiment, the optically transparent component has a tensile strength of >50 mPa. In another embodiment, the optically transparent component has a tensile strength of >100 mPa. In one embodiment, the optically transparent component comprises polyimide and the tag is selected from the group consisting of red fluorescent tags and near-infrared fluorescent tags. In another embodiment, the optically transparent component is a waveguide sensor formed of at least one polymer and at least one glass, where the glass is the waveguide material and the polymer is at least one material capable of preventing breakage and material loss of the device.

In one embodiment, the optically transparent component is a waveguide sensor with an aspect ratio that is less than 10:1. In another embodiment, the optically transparent component comprises at least one insertion material and at least one waveguide sensor such that the waveguide sensor is placed entirely in either a user's dermis or hypodermis. In one embodiment, the optically transparent component is comprised of a plurality of waveguides and insertion points into the skin that share at least one common optical emitter or detector. In another embodiment, the optically transparent component is one or more waveguide sensors located inside the lumen of a hollow needle placed into a user's skin. In one embodiment, the optically transparent component is least one microneedle sensor that less than 1 mm in length in insertion into a user's skin and which has a waveguide aspect ratio of >10:1.

In another embodiment, the optically transparent component is one microneedle element that penetrates skin by <1 mm and which rejects a percentage of autofluorescence from skin selected from the group consisting of 30, 60, 90, and 95%. In one embodiment, the analyte is NT-proBNP. In another embodiment, the analyte is pentameric C-reactive protein. In one embodiment, the analyte is monomeric C-reactive protein. In another embodiment, the analyte is IL-6. In one embodiment, the analyte is BNP. In another embodiment, the analyte is Troponin. In one embodiment, the optically transparent component is a planar waveguide.

In another embodiment, the device also includes a substrate, wherein the substrate is not a waveguide, and the substrate comprises an optical cladding that carries the planar waveguide. In one embodiment, the at least one optically measurable property is fluorescence intensity. In another embodiment, the at least one optically measurable property is the ratio between two fluorescence intensities from two tags. In one embodiment, the at least one optically measurable property is fluorescence wavelength. In another embodiment, the at least one optically measurable property is fluorescence lifetime. In one embodiment, the at least one optically measurable property has less than 30% signal loss over at least one day, or at least one week of continuous in-vivo operation. In another embodiment, the device further comprises a mechanical housing and the mechanical housing is less than 5 mm thick.

In one embodiment, the optical tag further comprises at least one photostabilizer molecule. In another embodiment, the device is capable of being applied to a user's skin and the sample fluid is interstitial fluid. In one embodiment, the device also includes at least one element to provide energy to the interface between aptamers and the sample fluid, to remove non-specific binding solutes from the aptamers. In another embodiment, the device is capable of being applied to a user's skin and the sample fluid is interstitial fluid, and further, wherein the at least one element to provide energy to the interface between aptamers and the sample fluid is located outside the skin.

In one embodiment, the device is capable of being applied to a user's skin and the sample fluid is interstitial fluid, and wherein the at least one element to provide energy to the interface between aptamers and the sample fluid is implanted into the skin along with the aptamers. In another embodiment, the energy is mechanical. In one embodiment, the energy is acoustic. In another embodiment, the energy is electrical field generated by an alternating charge of magnitude by a value that is selected from the group consisting of 1, 10, and 100 nC/cm².

In another aspect of the invention, a method for continually sensing at least one analyte in a sample fluid via measurement of the analyte is provided. The method involves at least one optically transparent component adapted for in-vivo placement. It also involves a plurality of aptamers bound to the optically transparent component wherein the aptamers bind to the analyte and carry at least one tag that changes in at least one optically measurable property when the aptamers bind to the analyte. Further, the method involves at least one optical source and detector coupled to the optically transparent component capable of measuring the optically measurable property of the aptamers. In addition, the method involves an optical source for exciting fluorescence of the at least one tag. The optical source is repeatedly but not constantly introduced to the optically transparent material with a duty cycle. Also, the duty cycle is a percentage of time during which the sensor is placed in the sample fluid, and the percentage of time is selected from the group consisting of less than 20%, 5%, 1%, 0.2%, 0.05%, and 0.01%.

### BRIEF DESCRIPTION OF THE DRAWINGS

The objects and advantages of the disclosed invention will be further appreciated in light of the following detailed descriptions and drawings in which:

FIG. 1 is a schematic of one embodiment of a device in accordance with principles of the present invention.

FIG. 2 is a schematic of one embodiment of a device in accordance with principles of the present invention.

FIG. 3 is a schematic of a prior art device.

FIG. 4 is a schematic of one embodiment of a device in accordance with principles of the present invention.

FIG. 5 is a schematic showing that surface area to volume and fluorescent signal can be achieved by using non-cylindrical geometries.

FIGS. 6A-6C are schematics showing that attachment chemistries can be used to minimize fouling. FIG. 6A demonstrates this chemistry with choline chemistry. FIGS. 6B and 6C demonstrate this chemistry with mixed surface charges.

FIGS. 7A and 7B are schematics showing that long-chain molecular brushes such as polyethylene glycol (PEG), or polyacrylamides, may be >1 nm or >5 nm in length then may also contain mixed charges and repel very large size foulants.

FIG. 8 is a schematic of one embodiment of a device in accordance with principles of the present invention.

FIG. 9 is a schematic of one embodiment of a device in accordance with principles of the present invention.

FIGS. 10A and 10B are schematics of embodiments of devices in accordance with principles of the present invention.

FIG. 11 is a schematic of example chemistry for stabilizing a fluorescent tag.

FIG. 12 is a schematic of one embodiment of a device in accordance with principles of the present invention.

FIGS. 13A and 13B are schematics of embodiments of devices in accordance with principles of the present invention.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers and other affinity-based probes. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “tag” is a molecule carried on an aptamer that has a measurable response as analyte binds to the aptamer, such as for example a fluorescent tag or quencher tag like that used in molecular beacons, or some other suitable tag that is measurable.

As used herein, the term “analyte” means any solute in a solution or fluid or sample fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “sample fluid” is the fluid containing the analyte.

As used herein, a “device” comprises at least one sensor based on at least one aptamer, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

As used herein, the term “fluorescent tag”, “tag”, and “fluorescent quencher”, and quencher means molecules which are like those used in molecular beacon laboratory assays. Examples of fluorescent tags include 6-FAM (carboxylflourescien), JOE, TET, HEX, and examples of quenchers include black-hole quenchers, DABCYL. These tags may also be referred to as “optical tags” more generally, as there are multiple types of optical emission beyond fluorescence such as phosphorescence, and because other optical properties such as optical absorbance magnitude or peak wavelength for optical absorption can also be measurable aspects of the tags. Tags may also include dyes that shift emission spectra, for example, pyrene dyes which have monomer emissions below 400 nm but when brought close together exhibit 485 nm emission.

As used herein, the term “folded aptamer” means an aptamer that along its length associates with itself in one or more locations creating a three-dimensional structure for the aptamer that is distinct from an “unfolded aptamer” that is a freely floating and oscillating strand of aptamer. Aptamers can also be partially folded or partially unfolded in structure or in time spent in the folded vs. unfolded states. Multiple folding configurations are also possible.

As used herein, the term “mixed charge monolayer” may be a protective monolayer and means a monolayer of at least partially vertically oriented molecules on a surface, comprising at least a first plurality of molecules with a first polarity of charge at or near their terminus facing the sample fluid, and at least a second plurality of molecules with a second polarity of charge at or near their terminus facing the sampling fluid, where the first polarity and second polarity are oppositely charged.

As used herein, the term “membrane” means a polymer film, plug of hydrogel, liquid-infused film, tiny pore, or other suitable material which is permiselective to transport of a solute through the membrane by solute parameters such as size, charge state, hydrophobicity, physical structure, or other solute parameters than can enable permiselectivity. For example, a dialysis membrane is permselective by passing small solutes but not large solutes such as proteins. Membranes as understood herein need not be multiporous, for example a nanotube or nanopore can act as a permiselective filter and is therefore considered part of a membrane as understood for the present invention. Permiselectivity can scale with the analyte, for example a membrane with a molecular weight cut-off of 50 kDa could be used to measure a 20-30 kDa protein but could still keep out cellular or other large content (globulins, fibrogen, etc.) and retain in aptamer that adequately large or physically structured such that permeability through the membrane is slow or nil.

As used herein, the term “optical core” and “optical cladding” or simply “core” and “cladding” refer to components of the term optical “waveguide”. A sample fluid may form an optical cladding. These components or materials confine light through total-internal reflection or reflection. If based on reflection, metals or photonic crystals can be used confine light. If based on total internal reflection the core must have a higher refractive index than the cladding.

As used herein, the term “optical source or emitter” and “optical detector” refer to optical components such as LEDs, lasers, and other optical sources of light and photodiodes, photomultiplier tubes and other optical detectors of light.

As used herein, the term “optical coupling component” refers to a component such as a lens or other optical component which couples light between an optical core and an optical source and/or detector.

As used herein, the term “at least one optically transparent component adapted for in-vivo placement” refers to adaption of an optically transparent component such that it is placeable via microneedle, indwelling, or implanted sensing formats into the body.

As used herein, the term “2×” means a difference of two times in magnitude. Similarly, the term “10×” means a difference of ten times in magnitude. The term “100×” means a difference of one hundred times in magnitude.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

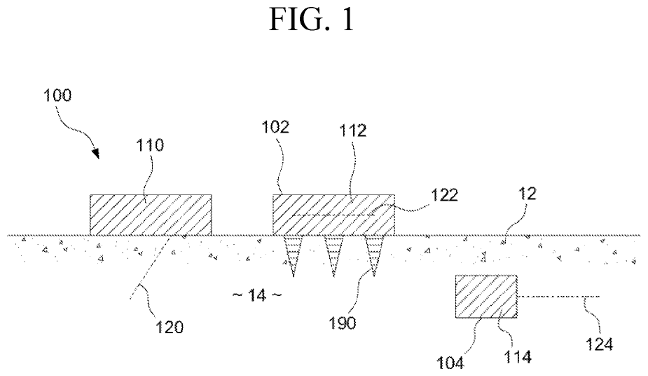

With reference to FIG. 1, in embodiments of the present invention devices 100, 102 and 104 are configured to measure at least one analyte in the body in interstitial fluid or blood, and are configured on skin 12 (100, 102) or implanted in the body (104). Each device has a housing 110, 112, 114, which may contain electronics and other required components for a sensing device. Each device has a waveguide sensor 120, 122, 124. In the case of waveguide sensor 120, the sensor is inserted into the skin. In the case of waveguide sensor 122, the waveguide sensor 120 is coupled to biofluid in the skin via porous or hollow microneedles 190.

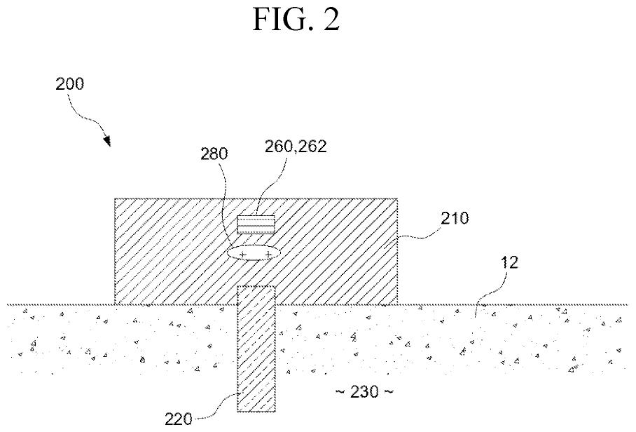

With reference to FIG. 2, where like numerals refer to like features, in embodiments of the present invention a device 200 carries a waveguide sensor 220 placed in a sample fluid 14 such as interstitial fluid or other fluid which contains analytes such as drugs, proteins, electrolytes or other important measurements. The device 200 has one or more optical coupling elements 280 between waveguide sensor 220 and source 260 and detector 262. Such elements 280 may be lenses, dichroic mirrors, gratings, monochromatic mirrors or other features to efficiently couple light between a waveguide, a source, and a detector. In alternate embodiments a plurality of sources 260 and detectors 262 may be utilized and in some cases may be directly coupled to waveguide sensor 220. In a device 200 elements 230, 232, 282 could also be a disposable component that matches up with reusable electronics and optics housed inside element 210. Not shown, but in an alternate arrangement, optical source 260 could be a light emitting diode or other optical source near the surface or skin 12 or inside skin 12 such that optical source simply illuminates sensor 220. The sample fluid or biofluid 230 in the example of FIG. 2 is interstitial fluid and/or blood depending on placement of sensor 200 in the skin (dermis, into a blood vessel, etc.).

With further reference to embodiments of the present invention, waveguides may be tapered in nature to obtain the benefits of tapered fibers in fluorescent waveguide biosensors, which include improved coupling of light between the waveguide and the fluorescent tags.

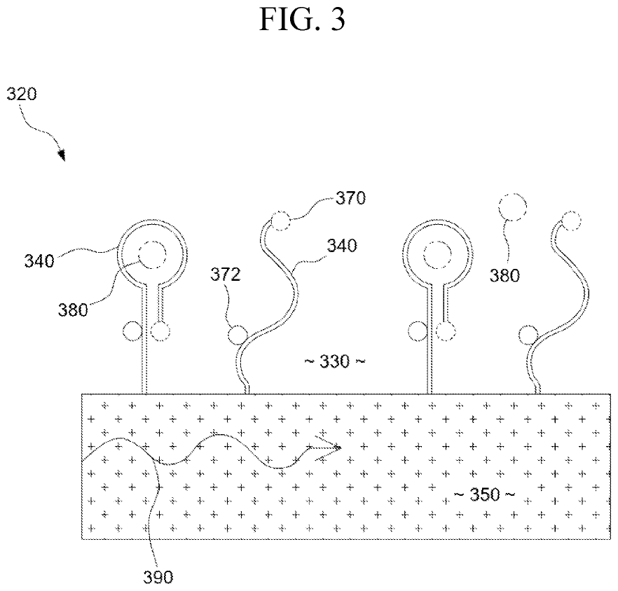

With reference to FIG. 3, a prior art device and sensing constructs are shown. While the shown constructs are highly simplified, they represent generally the type of constructs available after 20 years of prior work in optical aptamer sensors and molecular beacon aptamer assays. The aptamer 340 is specific to the analyte 380 such as a steroid hormone, a drug, a protein, etc. and when the analyte and aptamer bind the fluorescent tag 370 is brought close to a quencher tag 372 that quenches optical emission from tag 370. Because aptamers 340 are within roughly a wavelength of light distance from waveguide 350 they can be excited by light 290 through evanescent coupling or via light escaping waveguide 350 through refraction or scattering, and for aptamers 340 that are not bound to analyte 380 they will emit fluorescent light of a longer wavelength than light 390 that can then be coupled back into waveguide 350 and detected as described previously in FIG. 2. There are a large number of prior art examples, as taught for example in “Situma C, Moehring A J, Noor M A, Soper S A. Immobilized molecular beacons: a new strategy using UV-activated poly(methyl methacrylate) surfaces to provide large fluorescence sensitivities for reporting on molecular association events. Anal Biochem. 2007 Apr. 1; 363 (1): 35-45. doi: 10.1016/j.ab.2006.12.029. Epub 2006 Dec. 20. PMID: 17300739; PMCID: PMC2836515.” or for example as taught in “De Acha N, Elosúa C, Arregui F J. Development of an Aptamer Based Luminescent Optical Fiber Sensor for the Continuous Monitoring of Hg²⁺ in Aqueous Media. Sensors (Basel). 2020 Apr. 22; 20 (8): 2372. doi: 10.3390/s20082372. PMID: 32331372; PMCID: PMC7219322.” The aptamers generally but do not always require at least two tags (fluorescent and quencher tags) and a linker to bind the aptamer to the substrate, and any one of the tags or linkers can be attached at the 3′ end of the aptamer, the 5′ end of the aptamer, or at internal locations where the most common and widely utilized location is at an internal thymine site of the aptamer. Continuous optical aptamer sensors that are reagent free using a solid waveguide with at least one fluorescent tag and at least one quencher bound to at least one aptamer immobilized on the optical waveguide are disclosed in the prior art. Furthermore, colorimetric or other types of tags are possible so long as they are optically measured (for example, a tag that is changed in its absorption spectra as the aptamer binds to the analyte of interest).

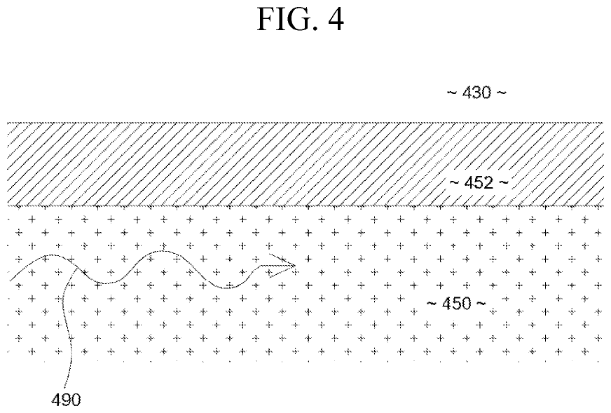

With reference to FIG. 4, in embodiments of the present invention and where like numerals refer to like features, the signal strength of emission from sensors 420 can be increased through one or more methods compared to the prior art. This is important because in some examples, one may go at great lengths to enable sensitive detection such as using expensive or bulky high-sensitivity optical detectors or using more complicated tapered optical fiber approaches. Furthermore, if the fluorescence signal is not adequately strong, then a higher excitation optical power for light 490 is used which can cause more rapid photobleaching of fluorescent tags. While such enhancements may be used with the present invention, they might not be absolutely required. In an embodiment of the present invention, waveguide 450 may be inherently porous or carry a porous coating 452, and aptamers 440 are immobilized in the pores. Such an embodiment generally requires, but is not necessarily limited to, an average pore size of at least 1 nm and ideally no greater than 100 nm, because at less than 1 nm it is difficult to enable mobility for most aptamers and greater than 100 nm light scattering (loss) increases. Pores may be at least one of >1, 3, 10, 30 nm and preferably less than at least on of <50, 100, 200, 500 nm in width (or diameter if circular). Materials 450 may and 452 may also be the same material (with 452 simply being a region into which pores are created that was originally material 450. As a nonlimiting example material 450 could be a cylindrical waveguide with 200 μm diameter and material 452 a 10 nm, 100 nm, or 1 μm, or 10 μm coating on material 450. Generally material 452 should have a refractive index greater than the refractive index of the sample fluid 430 such as interstitial fluid Alternately, entire waveguide 450 may be porous but generally only material 452 being porous is preferred to minimize device lag time (increased lag time can be caused by diffusion limited transport of the target analyte through a thick, porous material).

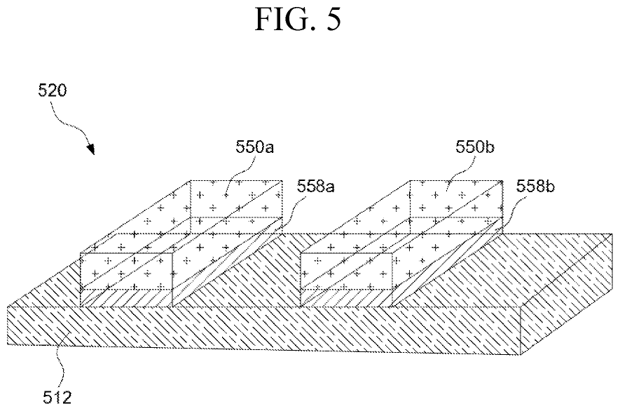

Embodiments of the present invention may also have multiple sensor waveguides each with its own distinct aptamers for measurement of analyte, as for example shown in FIG. 5, using multiple slab waveguides noted as waveguide a and waveguide b (550a and 552a, and 550b and 552b), or for example multiple fibers in a fiber bundle, or other suitable techniques. FIG. 5 further includes a substrate 512 such as PET or Kapton that carries an optical cladding 553, such as Teflon or Cytop or SiO2, and then a waveguide 550 of SiO2, PMMA polymer, SiON, Si3N4 that carries the fluorescently tagged aptamers, and optionally may include a porous portion of the waveguide 552 (not shown) that carries the fluorescently tagged aptamers such as porous SiO2 or a hydrogel.

With further reference to FIG. 4, several example embodiments are now taught in greater detail. Material 452 may be, for example, a polyacrylamide hydrogel coated by dip coating onto a glass or polymer optical fiber and with aptamers bound to the hydrogel by having aptamers that are acrydite modified. Example aptamers include those for mercury detection (CTTCTTTCTTCCCCTTGTTTGTTG), adenosine (ACCTGGGGGAGYAYYGCGGAGGAAGGT), for potassium or other suitable aptamers used as molecular beacons or chemically aptamers from vendors such as SomaLogic (Colorado) adapted into molecular beacon type switches. An example with molecular beacon tags for potassium detection is FAM-5′GATCTCAGGTAACCCTAACCCTAACCCCCTGAGATC3′-Dabcyl. Typically, such aptamers include a fluorescent tag and quencher that are readily commercially available through multiple sources and vendors, but in the case of mercury detection mercury can act as the quencher. Material 452 may be, for example, porous silica, and aptamers can be implemented with two or more fluorophores that enable Förster resonance energy transfer (FRET) which has an advantage that is can be less sensitive to loss of aptamers because the ratios of intensity between the two fluorophores can be measured. An example for FRET includes lysozyme detection using Biotin-TEG/TGG AAC TCA CTA CTC GAT TAG TGT ATG ACC TCT ATA TGA GAG CTT CTG AT/Cy3, Cy5/TAT AGA ATT ATA TTA TAT TAC GAG TAG TGA GTT CCA, where Cy3 is the donor fluorophore and Cy5 is the acceptor fluorophore as taught in Sapkota K, Dhakal S. FRET-Based Aptasensor for the Selective and Sensitive Detection of Lysozyme. Sensors (Basel). 2020 Feb. 9; 20 (3): 914. doi: 10.3390/s20030914. PMID: 32050422; PMCID: PMC7038949. FRET may also affect flourescence lifetime, which can allow calibration free operation because while fluorescent tags may photobleach and alter the response of the sensor, if instead fluorescence lifetime is measured then it will be insensitive to the effects of photobleaching. Therefore, the present invention enables at least one embodiment that is calibration free during use of the sensor in-vivo.

With further reference to embodiments of the present invention, multiple aptamers may for different analyte targets can be placed on the same sensor and used to multiplex sensing by having distinct fluorescent tags (for example blue fluorescing tag for a first analyte and a red fluorescing tag for a second analyte). Differing fluorescent spectrums can then be analyzed separately with miniature spectrometers or multiple photodetectors with spectral bandpass or notch optical filters or other suitable measures for multiwavelength measurement. Therefore, the present invention may measure a plurality of different analytes with at least one plurality of aptamers dedicated to each analyte.

In embodiments of the present invention materials 450, 452, or 550 or 552 may be for example primarily silica or have primarily silica on their exposed surfaces. Such surfaces may be prepared with piranha or other acids or oxygen plasma to create OH groups onto which molecules such as aptamers 340 can be covalently bonded using silane or other suitable chemistries for attachment on silica or other suitable oxides. Chemistries may include trichlorosilanes, (3-aminopropyl)triethoxysilane or -methoxysilane (APTES and APTMS) 3-mercaptopropyltrimethoxysylanes (MPTS) or other suitable attachment chemistries. Materials 450, 452 may also utilize polydimethylsiloxane or other silicone materials that can be oxidized to generate OH groups onto which molecules such as aptamers 340 can be covalently bonded. Dextran matrices may also be utilized for materials such as 452 and can be activated by adding functional groups to enable covalent binding of aptamers 340, but the presence of hydroxyl groups increases non-specific adsorption. The activation of dextran matrices with aspartic acid (leading to the formation of aldehyde groups) can help to reduce these non-specific interactions and adsorption. Dextran can then easily be attached to an aminated surface or self-contain amine residues to further react with and attach aptamers 340. Materials 450, 452 may also utilize poly(methyl methacrylate), and the surfaces of materials 450, 452, activated using hexamethylene-diamine to create primary amino groups to which molecules such as aptamers 340 can be attached. Gluaraldehyde may also be used to attach molecules 340 such as aptamers to poly(methyl methacrylate). Thiolated DNA can also be attached onto animated PMMA surfaces 450, 452 reacting with NHS-ester groups such as the ones from N-maleimidocaproyl-oxysulfosuccinimide ester (sulfo-EMCS) to achieve a covalent immobilization of molecules 340 such as aptamers. The present invention includes at least one aptamer attachment chemistry for which >90% of the aptamers will remain attached for at least 3 days in-vivo, testable in-vitro as 3 days of operation in serum at 33 or 37 degrees Celsius.

With further reference to FIG. 4, several example methods are now taught for increasing the density or coverage of available aptamers. Typical aptamer densities on a planar surface are limited to 10\hat{ }10 to 10\hat{ }13 aptamers per cm² of surface area. Embodiments of the present invention may utilize metal or oxide or other nanoparticles onto which aptamers are immobilized and the metal nanoparticle attached to materials 450 or 452 and the metal acting as a fluorescent quencher. In the example of an acrylamide hydrogel coating 452, when placed in interstitial fluid the hydrogel would have a refractive index greater than interstitial fluid (>1.35) because its refractive index when solid and without water is ~1.45. Materials 450 and 452 may include glasses such as silica glasses or other suitable glasses and polymers may include acrylics, cyclic olefin copolymer, polylactic acid, or other suitable polymers. Materials 450 and 452 may include for example cross-linked Poly(ethylene) glycol diacrylate or cross-linked polybetaine, which may or may not be further linked to aptamers depending on their placement and purpose as illustrated in FIG. 4 and other embodiments of the present invention. Porous glass can be made by phase separation of two glasses followed by a leaching technique, for example with alkali borosilicate and alkali aluminum borosilicate glasses which can be heated to 500-600 Celsius or greater to cause phase separation followed by etching in HF and HCl acids resulting in pore diameters of 1-10's nm and porous surface areas greater than 20 m²/g and up to 200 m²/g or more. Similar techniques are used to create porous Vycor glasses. Material 452 may also be aptamer coated nano-spheres of silica or polycarbonate that are adhered to material 450. For example, silica spheres could be dip coated in an acrylic adhesive and then the coating 452 plasma ashed in oxygen plasma to reveal most of the silica spheres area. Inverse templating is also possible, using for example polymer or wax nanospheres in silica sol-gel coating where curing the sol-gel curing at high temperatures can also burn off and vaporize the polymer nanospheres used for the templating.

With further reference to FIG. 4, in embodiments of the present invention and where like numerals refer to like features, parameters for surface area and signal strength are now taught. Assume that for aptamer packing densities of approximately 10¹⁰ to 10¹³/cm² the signal can be limited and require sophisticated electronics. Further assuming as an example a 100 μm diameter fiber, the as taught for FIG. 3, the surface area per unit length in cm would be 2*3.14*50E-7 cm*1 cm=31.4E-6 cm² per cm length of sensor waveguide. A hydrogel coating 452 that is only 100 nm thick and with the equivalent of 1 layer of aptamers at similar densities mentioned above at every 5 nm (which is ample spacing) would have 20× higher fluorescent signal. This is a simple example which can be scaled in either direction using this or other examples taught above, such that the present invention enabled a fluorescent signal that is at least one of 2×, 10×, 50×, 200×, 1000× greater than the equivalent maximum fluorescent signal for a smooth planar 100 μm diameter waveguide as illustrated in FIG. 3. Surface area to volume is another way to interpret the present invention. Further assuming as an example a 100 μm diameter fiber that is 1 cm long, as taught for FIG. 3, the surface area per volume would be 2*3.14*50E-7 cm*1 cm=31.4E-6 cm² per 3.14*(50E-7 cm)\hat{ }2*1 cm=7.85E-11 cm³ or 31.4E-6/7.85E-11=40,000. Using the above taught example, the surface area to volume would be 20× larger. Therefore, the present invention enables a surface area to volume that is greater than 40,0000 by at least one of 2×, 10×, 50×, 200×, 1000× greater than the equivalent surface area to volume of a smooth planar 100 μm diameter waveguide per cm of waveguide length. While the present invention is not limited to a 100 μm diameter cylindrical waveguide, this example will be used as a testable metric for proper interpretation of the specifications and claims. For example, if comparing to art with a 10 μm diameter waveguide, then the same calculations used above would be implemented except for substituting 10 μm diameter waveguide for 100 μm diameter waveguide. Slab or other types of waveguides are similarly substitutable.

With further reference to embodiments of the present invention surface area to volume and fluorescent signal can be increased through use of fiber bundles or bundles of waveguides (not necessarily cylindrical). As a non-limiting example consider one specific example of many used in endoscopic imaging, as taught in “Orth A, Ploschner M, Wilson E R, Maksymov I S, Gibson B C. Optical fiber bundles: Ultra-slim light field imaging probes. Sci Adv. 2019 Apr. 26; 5 (4): eaav1555. doi: 10.1126/sciadv.aav1555. PMID: 31032405; PMCID: PMC6486219.” A fiber bundle can be manufactured for example using two types of glasses and one glass phase etched away (as taught in previous examples, but the fibers would be formed, fused, drawn to decrease diameter, then etched). Similar approaches could be used for polymer fibers (fuse or bundle in a second polymer, draw to decrease diameter, then dissolve away the second polymer, or the second polymer could remain because it is a hydrogel polymer for example). Regardless of fabrication method, consider the 100 μm diameter fiber example but applied to a 100 μm fiber bundle of fibers of 5 μm radius, which in a hexagonal array has a packing density of 0.9069 or less. The number of fibers in the bundle is 3.14*(50E-4 cm)\hat{ }2*0.9069/(3.14*(2.5E-4 cm)\hat{ }2))=362 fibers. The surface area per cm of the fibers in the bundle is 2*3.14*2.5E-4 cm*1 cm=1.57E-3 cm2 per cm. The resulting increase in surface area compared to a single solid fiber of 100 μm diameter is therefore (1.57E-3 cm2/cm)/(31.4E-6 cm2/cm)=50× larger. Fibers can be made as small as just several μm, such as 2 μm, which could further increase the surface area. Tight packing of the fibers in the bundle could create lag time, so the fibers can be separated for example by immersion and bending in a tank with cellulose or other fibers that can work in between the fibers, or for example the fibers can be infused with a hydrogel (polymer) that swells upon contact with fluid and provides space in between fibers for analyte diffusion. This spacing is also important to prevent abrasion between fibers. Generally, embodiments of the present invention may include a plurality of fibers in a bundle with at least 1 μm separation between the fibers.

With further reference to embodiments of the present invention and FIG. 5 where like numerals refer to like features, surface area to volume and fluorescent signal can be achieved by using non-cylindrical geometries. For example, the sensor strip on a Freestyle Libre glucose monitor which is placed in the skin is 0.4 mm wide. A slab waveguide can be fabricated that is 10 μm thick and 0.4 mm wide. A 100 μm fiber has a circumference of 2*3.14*50 μm or 300 μm. Therefore, the slab waveguide has a much greater surface area to volume ratio. Slab waveguides can be made from polymers or glasses or polymers coated with glasses. Low temperature deposition even on planar and flexible substrates can be achieved using sol-gel (such as TEOS) or plasma-enhanced chemical vapor deposition (such as SiO2 or Si3N4) or low temperature deposition such atomic layer deposition (Al2O3, etc.). For most substrates 512 such as Kapton, a cladding 558a, 558b is required as illustrated in FIG. 5a for two or more slab waveguides 550a, 550b, with suitable claddings being lower refractive index polymers and glasses as understood by those skilled in the art of planar or slab waveguides.

With further reference to embodiments of the present invention aptamers can contain a plurality of fluorescent tags to increase signal, and can be achieved by tagging the aptamers at plurality of locations where the fluorescent tags are at least 5 or at least 10 nm in distance from each other. The distance between DNA bases is 3.4 nm, such that a distance of 3-4 bases is sufficient, or the fluorescent tags can be attached via a chain or network of molecules attached to a part of an aptamer. Single or multiple quenchers can be used so long as they are in close proximity the fluorescent tags when the aptamer is bound or unbound from the analyte.

With further reference to embodiments of the present invention photobleaching can be reduced by increasing the density or coverage of aptamers. For example, as taught previously the present invention can enable a fluorescent signal that is at least one of 2×, 10×, 50×, 200×, 1000× greater than the equivalent maximum fluorescent signal for a smooth planar 100 μm diameter waveguide. If for example a fluorescent dye such as CY3 or CY5 photobleaches by 30% over 1 hour on a planar waveguide (which is an achievable result), and the fluorescent signal was increased by 200× using one or more methods as taught herein, then the optical power for excitation light 490 can be reduced by as much as 200×, such that 30% photobleaching does not occur until as many as 200 hours (>8 days). Therefore, the present invention includes a device with less than 30% signal loss over at least one day, or at least one week of continuous in-vivo operation.

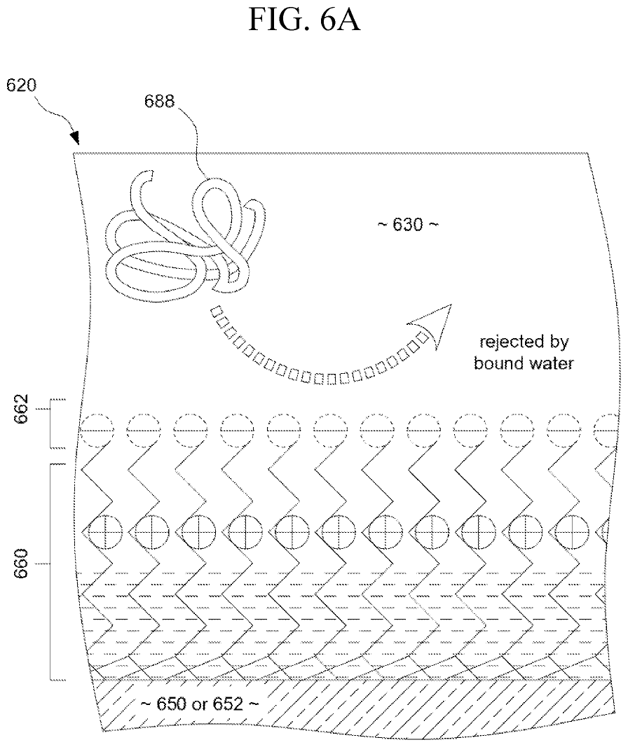

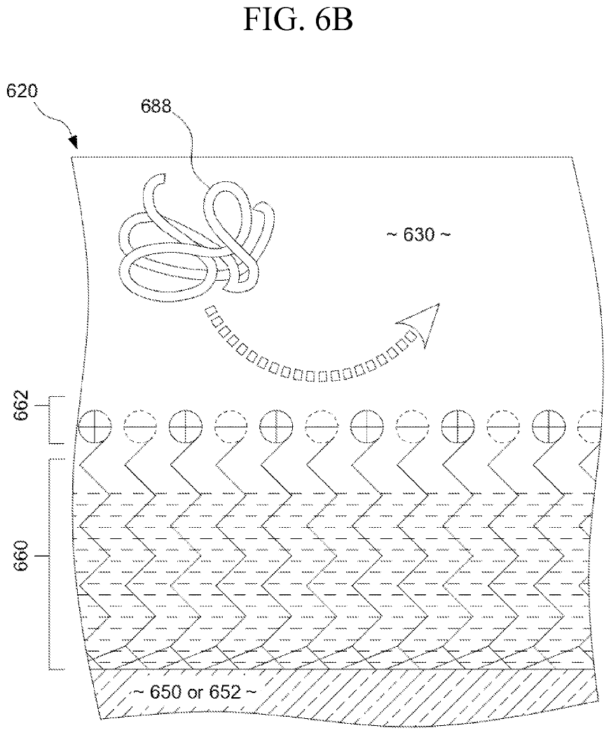

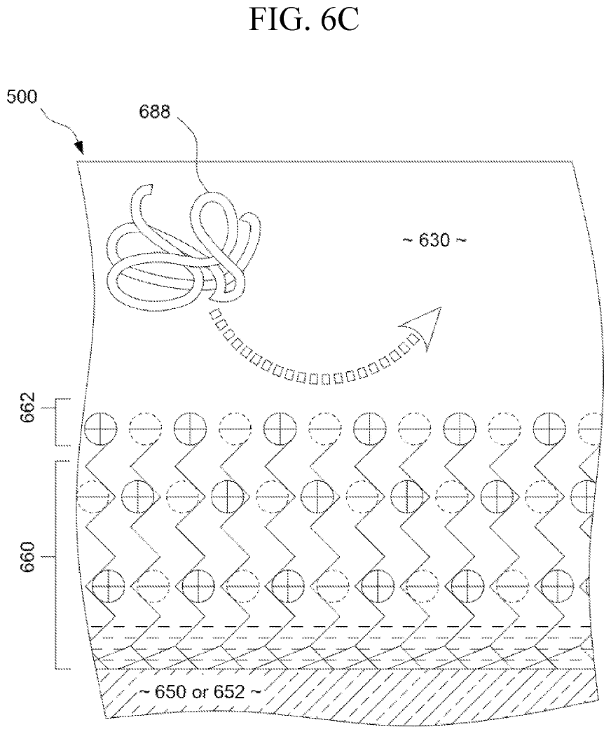

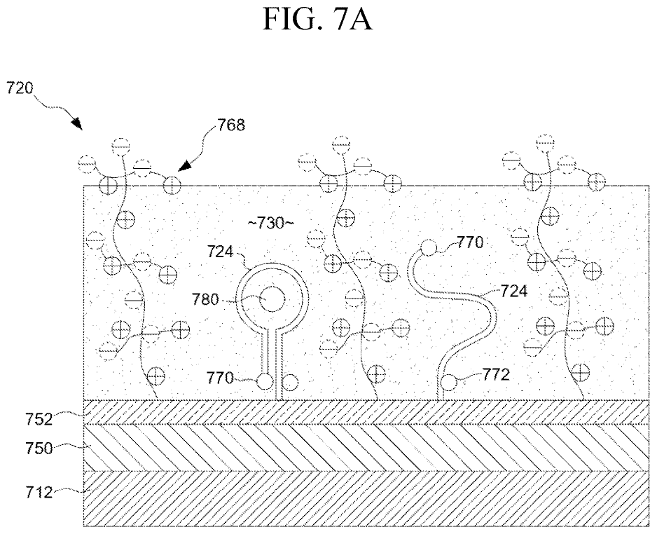

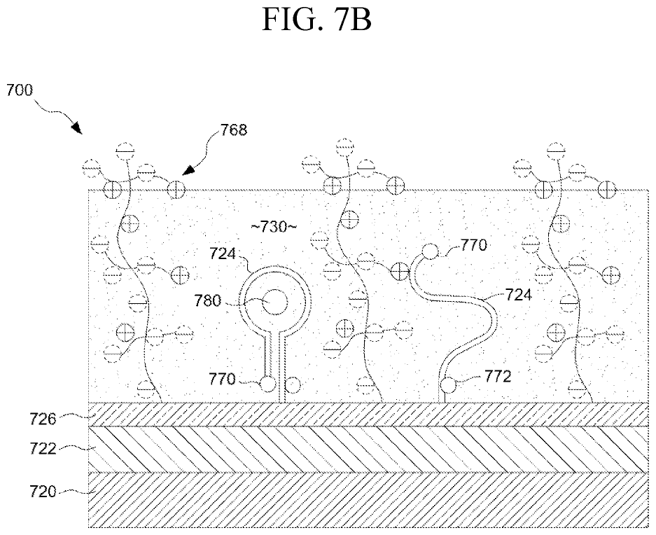

With further reference to embodiments of the present invention, a challenge with aptamer sensors is that when placed into initial operation the sample fluid 130, over a period of minutes to hours to days fouling can degrade the sensor response. Optical aptamer sensors have not yet been demonstrated in-vivo and the resulting longevity problems that will occur in-vivo have not yet been resolved in in-vitro experiments, especially for experiments that rely on polyethylene glycol methods for antifouling which oxidizes and degrades in-vivo. As illustrated in FIGS. 6 and 7, attachment chemistries can be used to minimize fouling by foulants 688, ideally using charge or mixed charge to maximize bound water at the surface 662 or inside the surface chemistry 660. For example, a zwitterionic chemistry is demonstrated in FIG. 6a, using for example choline chemistry. Mixed surface charges are also possible as illustrated in FIG. 6b, 6c, and in one embodiment, the monolayer is composed of zwitterions where one molecule is terminated in a phosphorylcholine group and the other molecule is terminated in a sulfobetaine group. Negative charges are preferred nearest the surface to repel most foulants which are also negatively charged. Surfaces can have net charges but too strong of a net charge can both increase fouling by attracting oppositely charged foulants and can impart electrical force on the aptamer which can in some cases negatively affect sensor response. Aptamers and foulants typically, but not always, have negative charges. For example, if the mixed charge surface had a density of 4 monolayer molecules per nm² and 40% were positively charged and 60% were negatively charged at or near their terminus facing the sample fluid, then the monolayer would have a net charge per nm² that is negative and which has 0.2 charges*4 molecules/nm²=0.8 charges per nm². This would satisfy a mixed charge monolayers with a net charge that is at least one of less than 50% of an equivalent of a net charge of 1 charge per nm² (it is less than 1 charges per nm²). In another embodiment, a net charge is preferably negative such that most foulants and aptamers are slightly repelled from the surface. For example, a net charge could provide a surface potential at the sample fluid of −20 mV or at least −10 mV but not more than-30 mV which is shown to work well for aptamer switches on surfaces. As illustrated in FIG. 7, long-chain molecular brushes such as polyethylene glycol (PEG), or polyacrylamides, may be >1 nm, >2 nm, or >5 nm in length then may also contain mixed charges and repel very large size foulants. Furthermore, as illustrated in FIG. 7b because in some cases Debye length extension is known to improve aptamer binding affinity and sensor response, the near-aptamer portions of such long-chain molecular brushes can be negatively charged to extend the Debye length. As a result, Debye length is extended similar to that achieved within a nanoporous structure as taught and possible in other embodiments of the present invention. Therefore, the present invention may further include a plurality of molecular brush molecules interspersed between aptamers. Therefore, the present invention may further include a plurality of molecular brush molecules interspersed between aptamers that have a net charge near the aptamers and which extend the Debye length from the boundary layer with the mixed charge monolayer by at least 2× in length. Attachment chemistries for aptamers and antifouling layers on glasses may include silanes, phosphonic acid or other suitable chemistries optimal for each surface type as understood by those skilled in the art of monolayer chemistry. Polymers can functionalized such as polymeric biomaterial, poly(2-hydroxyethyl methacrylate) hydroxyl group in the side chain of the polymer, polyethylene surface via chain transfer to the polymer by free-radical polymerization, or other techniques as for example taught in Abshar Hasan & Lalit M. Pandey (2015) Review: Polymers, Surface-Modified Polymers, and Self Assembled Monolayers as Surface-Modifying Agents for Biomaterials, Polymer-Plastics Technology and Engineering, 54:13, 1358-1378, DOI: 10.1080/03602559.2015.1021488.

With further reference to embodiments of the present invention, the sensors may be protected by one or more external membranes or hydrogels which can be separate from the sensors surface (such as placing the sensor in the lumen of a microdialysis tube) or for example by forming the membrane onto the sensor surface itself, for example by dip-coating and UV cross-linking of polybetaine or other hydrogels. Polymer waveguides or porous glass waveguides may also prevent fouling and foreign body response by being co-mixed with a slowly-eluting drug such as dexamethasone, with non-limiting examples of polymers including Poly(ε-caprolactone), polyethylenimine, dense chitosan, Poly(d,l-lactic-co-glycolic acid), or some of the other polymer materials as taught herein.

With further reference to embodiments of the present invention, many sensors, such as those formed on glass waveguides, could risk breaking off inside the body and therefore produce long-term adverse effects at the implantation site for the sensor. While sensor insertion can use a slotted inserter like that used in the Freestyle Libre glucose monitor, insertion is not the only event that could damage or break the waveguide sensor. Furthermore, sensor surfaces can be sensitive to abrasion or pressure (e.g. can be degraded, or can impart motion or pressure artifacts on the sensor signal by pressing against the aptamers). Therefore, sensors of the present invention may include woven metal jackets or other protective schemes or mechanically protective elements like those used in endoscopic imaging tubes except, unlike endoscopy, the jacket or protective scheme must be porous to the analyte. Preferred metals may include stainless steel, titanium and Co—Cr alloys, and may be further coated with anti-fouling chemistry or hydrogels to minimize foreign body response. Therefore, sensors of the present invention may include at least one protective sheath or mechanically protective element that is porous to the analyte. A microdialysis tube is also an example of a mechanically protective element that is porous to the analyte.

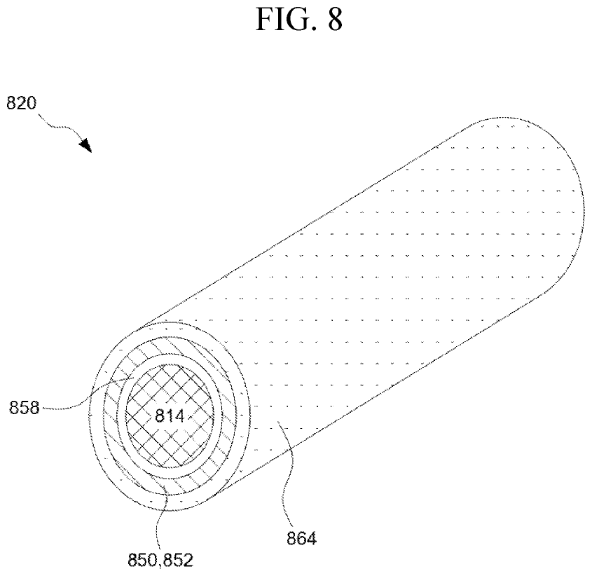

With further reference to FIG. 8, in embodiments of the present invention and where like numerals refer to like features, another embodiment is taught to promote safe insertion and removal of a waveguide sensor 820, compared to for example an all-glass fiber that could more easily breakoff inside the body. The waveguide sensor 820 is formed from a non-breakable material 814 such as a Kapton fiber or even more preferably a 200 μm diameter Ta wire, that is then further coated with a cladding material 858 such as SiO2 or even lower refractive index aerogels or fluorinated glasses or inorganic or polymers or fluoropolymers that are for example 10 μm thick, followed by a coating of waveguide materials 850, 852 as previously taught (material 852 can be optional) such as alumina or Ta2O5 or SiO2 or polymers that are of higher refractive index than element 858 and are for example 50 μm thick, and a protective membrane 864 as previously taught that is 40 μm thick such that the total diameter of 400 μm is achieved. The layers of such as device can be made even thinner/smaller to promote ease of insertion into the body and may have a sharp leading edge to enable ease of piercing the skin. Coupling of light into a cylinder can be achieved simply by illuminating it knowing that some light is lost into element 814 or for example a circular diffraction grating can be used with laser light source to shine a dominant 1^{st} order diffraction mode only into the cylinder 852 at the critical angle for propagation of light in waveguide 850, 852.

With further reference to embodiments of the present invention and where like numerals refer to like features, a waveguide sensor 820 can be formed of an organic material such as polymer that is less likely than glass to fracture and break (because it is more flexible, deformable), and the material may have a high tensile strength of >50, or at least >100 MPa. Suitable materials include, for example, polyethylene terephthalate, polycarbonate, glass reinforced polymers such as glass-fiber reinforced polycarbonate or polyethylene terephthalate, or polyimide. A glass-reinforced fiber may require index matching to minimize optical scattering if the glass fibers are >10's to 100's nm in size, for example using flint glass fibers (refractive indices near 1.6) with polycarbonate or polyethylene terephthalate. Polyamide such as Kapton has poor optical transparency especially in the blue and green wavelength regions, and therefore use of a red tag such as Texas red (sulforhodamine 101) and a DABCYL quencher could allow at least partial compatibility with Kapton. Kapton waveguide sensors 820 may require shorter lengths (such as <3 mm, or even <1 mm) to further promote proper transparency. These higher tensile strength materials may carry molecules such as aptamers directly or as taught for embodiments of the present invention have coatings that carry aptamers. Even longer wavelength fluorescent dyes may be preferred such as near-infrared dyes IRDye 800CW and IRDye 800RS.

With further reference to embodiments of the present invention and where like numerals refer to like features, a waveguide sensor can be formed of at least one polymer and at least one glass, where the glass is the waveguide material and the polymer is at least one material to prevent breakage and material loss inside the body (for example as previously taught for FIG. 5).

With further reference to embodiments of the present invention aspect ratio can have a strong impact on potential breakage of a waveguide, and the present invention may for example use a waveguide that is at least less than 1000 μm in length and at least greater than 100 μm in diameter or width such that waveguide at least has an aspect ratio that is less than 10:1.

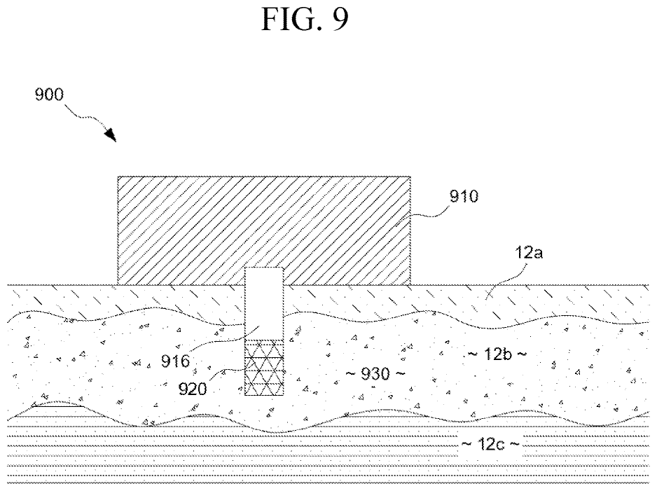

With reference to FIG. 9, where like numerals refer to like features, in embodiments of the present invention while the prior art has created devices with in-vitro capability for in-vivo placement with sensing they are not readily usable with skin implantable format in terms of reliable insertion depths. For example, for most analytes to be measured, implanting the sensor waveguide 920 in the epidermis 12a is not preferred to placement in the dermis 12b or hypodermis/subcutaneous layer 12c. Any portion of waveguide 920 not at its proper depth can cause less accuracy of measurement and/or increased measurement lag times. Therefore, the present invention includes at least one waveguide sensor 920 carried by at least one insertion material 916 such that waveguide sensor 920 is placed entirely in at least one of the dermis 12b or hypodermis 12c. For a commercially successful device this condition of placement in the proper layer of skin would occur at least 90% of the time such that 9 out of 10 devices 900 would function properly. For example, material 916 could simply be the same material as material 920 but material 916 is only 0.2 or 0.5 mm in length and material 920 is 0.1 mm in length and carries a plurality of aptamers. Material 916 and material 920 could also be different materials, for example material 916 being a polymer optical fiber and material 920 a porous glass optical fiber as taught in other embodiments of the present invention with material 916 and 920 bonded by for example optically clear adhesive or epoxy as used in bonding optical materials such as plastics and glasses.

Embodiments of the present invention may be made shelf stable by preserving in trehalose solution which is then dried under vacuum or other suitable techniques. Embodiments of the present invention may be made shelf sterile by techniques such as e-beam, gamma, or other suitable sterilization techniques.

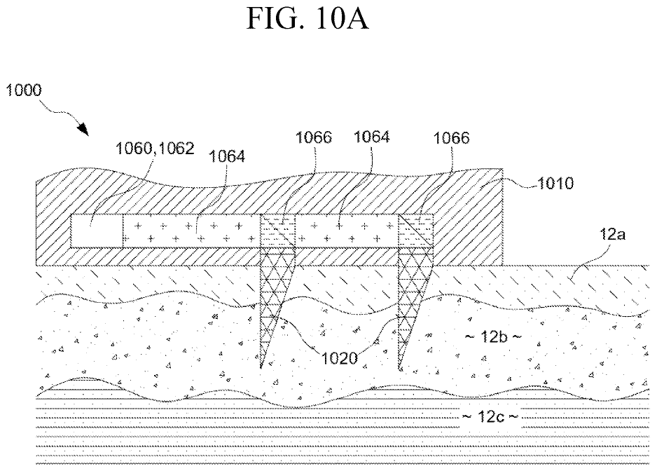

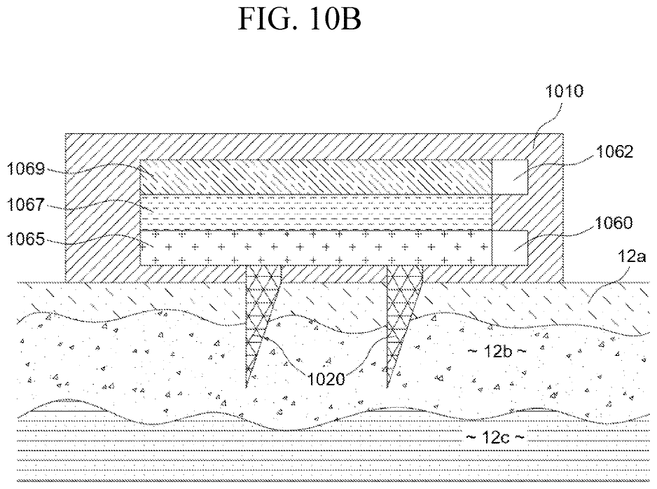

With reference to FIG. 10, where like numerals refer to like features, in embodiments of the present invention a plurality of waveguides and insertion points into the skin are included for a device 1000 of the present invention. FIG. 10a includes a plurality of waveguide sensors 1020 for the same analyte such that at least one sensor 1020 makes reliable contact with the dermis 12b or hypodermis 12c. Waveguides 1020 are coupled via 50/50 beam splitters 1066 to coupling waveguide 1064 that is further coupled to optical emitter and detectors 1060, 1062. As a result, the embodiment demonstrates a plurality of waveguides and insertion points into the skin that share at least one common optical emitter or detector. In an alternate embodiment in FIG. 10b, element 1060 is an emitter, element 1065 is a backlight plate like used in liquid crystal displays to couple light into elements 1020, element 1067 is a thin film filter that blocks light from element 1060 (such as blue light) but which passes fluorescent signal from aptamers on elements 1020 (such as green light), element 1069 is a dye-doped waveguide that converts light (such as green to red light), and element 1062 is a detector. Alternately element 1069 could be a diffractive or refractively coupled waveguide to guide light to detector 1062 or element 1069 could be replaced in part or entirety by element 1062. In practice, emitter 1060 can introduce blue light into element 1065 which is further coupled into elements 1020, which then provide a green fluorescent signal from aptamers back through element 1065 and through element 1067 where yellow or red fluorescent dye in 1069 absorbs said green signal and re-emits it as longer wavelength light such that it is waveguided onto detector 1062.

With reference to FIG. 10, where like numerals refer to like features, in an alternate embodiment, elements 1020 may have different aptamers and target different analytes. Aptamers can be patterned distinctly onto distinct elements using methods such as drop casting, UV attachment or crosslinking, or other suitable methods.

Embodiments of the present invention may embed one or more waveguide sensors in the lumen of a hollow needle placed into the skin. For example, a NanoPass MicroJet silicon microneedle array which are 600 μm long may be utilized as taught in part in US patents—U.S. Pat. Nos. 6,533,949; 7,648,484; 7,850,657; 8,454,844; 7,998,119; 8,007,466; 7,285,113; 7,588,552. The silicon can be thermally oxidized including inside the silicon needle lumen to a thickness of 5 μm with oxide of refractive index 1.46 then coated via atomic layer deposition with aluminum oxide with 5 μm to form a higher refractive index of 1.76 which then may act as a waveguide and carry a plurality of aptamers. The lumen can be further filled with a hydrogel such as agar or polybetaine to reduce fouling or to carry aptamers.

With reference to FIGS. 1, 9, and 10, in embodiments of the present invention the waveguide sensors utilized can be very short such that their ability to reject autofluorescence is diminished. Two example approaches are provided to deal with such autofluorescence. First, consider the example dimension of microneedles such as those by Nanopass which are 600-700 μm deep and at their base as much as 200 μm wide. Such a geometry would pick up autofluorescence from tissue and fluid surrounding the waveguide. In this first example, the waveguide, using one more previously taught embodiments, is coated on the outside of the microneedles for example as 50 μm thick silica. In this example the waveguide aspect ratio is not 600 to 200 μm or 3:1 but rather it is 600 to 50 μm or more than 10:1. Therefore the present invention includes at least one microneedle sensor that is <1 mm in length in insertion into the skin and which has a waveguide aspect ratio of >10:1. In a second example, such for example FIG. 9, element 916 is coated with an optional cladding and with a light absorber such as carbon black paint or another type of absorptive material which would block or absorb autofluorescent light. If for example elements 920 and 916 were 100 μm in diameter (r=50) then element 920 may be 200 μm long and element 916 may be 400 μm long.

With reference to embodiments of the present invention with microneedles as illustrated in FIG. 1 as elements 190 they may not be long enough and narrow enough to act as a waveguide. A waveguide is desired to overcome background interference from autofluorescence in the sample fluid. However, if the optical material 452 is porous and has adequate density or number of aptamers the resulting signal can overcome autofluorescence even without use of a waveguide. Therefore, the present invention does not necessarily require use of a waveguide and simply requires at least one optically transparent component that carries the aptamers. Furthermore, any waveguide or material 450 or 452 can be partially protected externally autofluorescence by using an external light blocking protective layer (not shown) such as dye-stained membranes or membranes impregnated with pigments or other Ti coated track etch membranes or other suitable materials.

As a result, the present invention further includes at least one microneedle element that penetrates skin by <1 mm and which rejects at least one of 30, 60, 90, or 95% of autofluorescence from skin.

One of the significant challenges that exists for in-vivo biosensing with fluorescently tagged aptamers is photobleaching and poor longevity. While most imaging experiments with fluorescently tagged DNA and most in-vitro assays with fluorescently tagged DNA (such as molecular beacons) require seconds to minutes of operations, most continuous in-vivo measures will require days of continuous measurement if not at least one week and ideally two weeks of measurement. This places a major challenge with respect to photobleaching. As recently as 2023, Soh and colleagues state in the following paper “Continuous optical detection of small-molecule analytes in complex biomatrices,” Amani A. Hariri, Alyssa P. Cartwright, Constantin Dory, Yasser Gidi, Steven Yee, Kaiyu Fu, Kiyoul Yang, Diana Wu, Ian A. P. Thompson, Nicolò Maganzini, Trevor Feagin, Brian E. Young, Behrad Habib Afshar, Michael Eisenstein, Michel Digonnet, Jelena Vuckovic, H. Tom Soh, bioRxiv 2023.03.03.531030; doi: https://doi.org/10.1101/2023.03.03.531030, “We selected a Cy3 fluorophore and an Iowa Black quencher to label the aptamer and displacement strands, respectively, in order to achieve long-term measurement with minimal fluorescence background or photobleaching.” At 0.5 μW of laser power for the optical source, the sensors lost 10% of their signal over 30 minutes, which is insufficient for a device that would operate for at least one day, a week, or two weeks. A first solution to this challenge is to periodically activate the laser source at the cost of reduced measurements. For example, assume the laser or LED or other optical source must be active for 500 ms for each measurement. Assume less than 1 hour of continuous exposure to the optical source causes 20% photobleaching (signal loss) and that is determined to be an acceptable amount of signal loss during in-vivo operation. 1 hour divided by 500 ms is 7200 potential measurements, and if the analyte is measured only every 5 minutes, this represents 7200*5/60 or 600 hours of measurement. Therefore, the present invention may used for periodic or intermittent source activation for less than 1 minute, less than 10 s, or less than 1 s duration to measure the sensor at intervals greater than 1 minute and achieve at least one day, 3 days, 7 days, or 2 weeks of continuous operation. Therefore, the present Invention includes a duty cycle for the optical source, and wherein the duty cycle is less than 20%, 5%, 1%, 0.2%, 0.05%, or 0.01% of time during which the sensor is placed in the sample fluid.

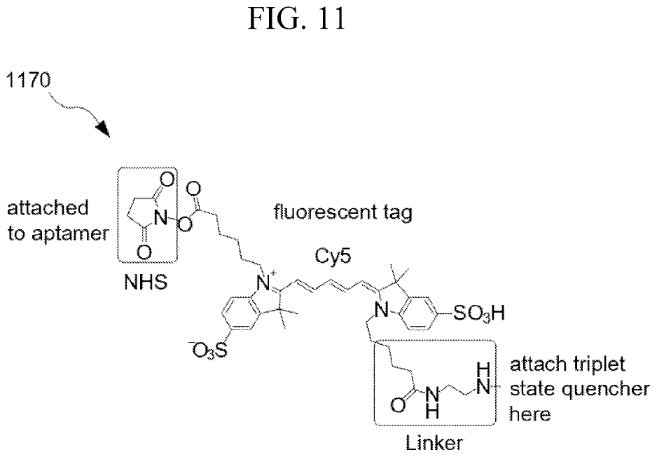

With further reference to embodiments of the present invention, the fluorescent tag itself can be further chemically stabilized. For example, aptamers can be suspended in water and double-distilled (dd) H₂O and adjusted to 50 μM in 50 mM potassium borate buffer (pH 8.1) with 200 mM KCl. The aptamers can then be labeled by adding a fivefold molar excess of NHS-reactive fluorescent tags resuspended in dimethyl sulfoxide (DMSO) in a 10-μl reaction and incubating at 37° C. for 30 minutes. An example of such chemistry is shown in FIG. 11, wherein cyanine 5 (Cy5) is stabilized with a triplet state quencher such as cyclooctatetraene, 4-nitrobenzyl alcohol, or 6-hydroxy-2,5,7,8-tetramethylchroman-2-carboxylic acid. The above example can reduce photobleaching by up to 50-fold. Therefore, the present invention may include at least one photostabilizer molecule bound to the fluorescent tag.

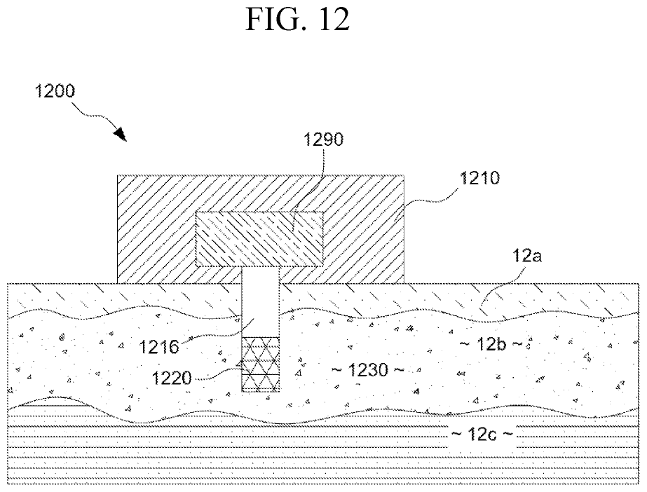

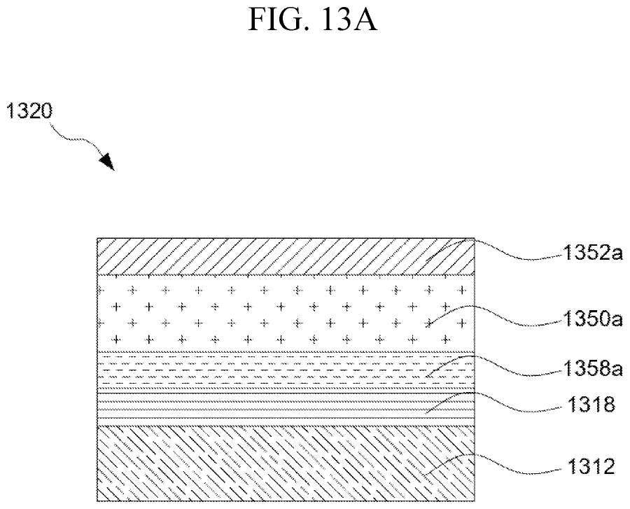

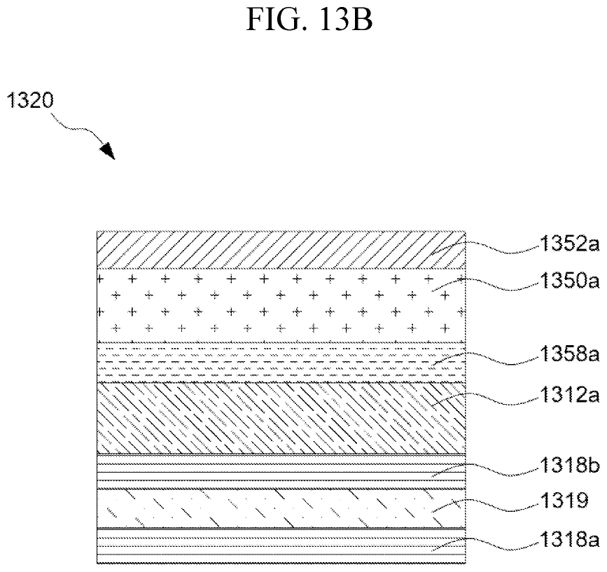

With further reference to embodiments of the present invention, a remaining challenge for an optical aptamer sensor is non-specific binding of solutes in the sample fluid to the aptamers. While embodiments of the present invention teach ways to minimize fouling of the sensor surface (for example FIG. 6 or 7), aptamers are often unprotected from non-specific binding to solutes such as those found in interstitial fluid. Non-specific binding can be a simple as charge interaction between peptides or proteins and the negatively charged aptamer phosphate backbone. In embodiments of the present invention, FIGS. 12 and 13 provide non-limiting examples of providing energy to the sensor/fluid interface to remove non-specific binding which is normally weak enough that it is removable. FIG. 12, for example, includes a mechanical vibrating (such a piezo electric crystal) or acoustic element such as an ultrasound source 1290 which can impart an acoustic or mechanical wave or vibration ranging from kHz to MHz to GHz to the sensor 1220 to prevent non-specific binding. Direct vibration, fluid flow vortices, and other disturbances at the sample/sensor interface can be created to remove non-specific binding. Geometries and coupling methods suitable for the present invention are possible for example by using a ultrasonic wire bonder, and instead of using a gold wire through the wire bonder needle, instead placing the optical fiber through the wire bonder needle. With reference to FIG. 13, the energy to the sensor/fluid interface can also be applied locally. For example, a gel-pad electrode can be applied to the skin surface and a counter electrode 1318 placed beneath the optical waveguide 1350a to create a capacitively coupled electrical circuit through the waveguide 1350a. A 1's, 10's 100, 1000's Hz or higher frequency electric field can be applied though the optical waveguide, with strengths of electric field similar to that used in electrochemical aptamer sensors on gold electrodes (100's mVs), to electrically modulate (oscillate or move) the negatively charged aptamers and remove non-specific bonding solutes. For example, if capacitive coupling is utilized, the required electric field can be derived using a conventional electrochemical aptamer sensor as an example. For example, an alky thiol monolayer blocking layer with a dielectric constant of ~2.6 and ~0.7 nm thickness can have an electrical capacitance C=e*eo*A/t of C=2.6*8.854E-14 F/cm/7E-8 cm=3.3 μF/cm². The electrical double layer capacitance is so large that it is negligible in this calculation (due to thinness of the double layer in high salt conditions such as biofluids and the very high dielectric constant of water). In electrochemical aptamer sensors typically are oscillated in electric field using 10's mV in square wave voltammetry. Therefore, the charge provided at the sensor surface with the biofluid can be 10's mV*3.3 μF/cm²=10's to 100's nC/cm². Layers 1358a and 1350a could have an electrical capacitance that is multiple of orders of magnitude smaller than 3.3 μF/cm² due to a greater thickness of layers 1353a and 1350a. As a result, if for example the capacitance was 100× smaller, then the voltage applied could be 100× larger (1's or 10's V) to achieve similar electric field modulation of the aptamers. The voltage drop or frequency applied would ideally be limited to across materials 1358a, 1350a such that electrical effects on skin or the body are mitigated. The present invention may therefore further include an electric field generated by an alternating charge of magnitude that is at least one of 1, 10, or 100 nC/cm². Generally, to keep the aptamers in close proximity to the electric field, which diminishes with distance into a fluid (Debye length), ideally the aptamers are attached such that they are in close proximity to the electric field as well, similar to electrochemical aptamer sensors. This therefore may require anti-fouling chemistry which is thinner such as illustrated in FIG. 6. A DC electric field is also possible for non-specific binding solutes with a strong charge (they can be repelled by the DC field). As illustrated in FIG. 13b, a local component for inducing energy at the sensor/fluid interface is also provided, such as a ZnO or LiNbO3 piezoelectric film 1319, which can be 100's of nm or 100's of μm thick and have two or more electrodes 1318a or 1318b to enable piezoelectric vibration of sensor 1320.

The present invention involves a device that can be applied to the skin. It uses aptamers to analyze a sample fluid such as, for example, interstitial fluid. The device may further comprise at least one element to provide energy to the interface between aptamers and the sample fluid, to remove non-specific binding solutes from the aptamers. In one embodiment, the device uses an element to provide energy to the interface between aptamers and the sample fluid is outside the skin. In another embodiment, the element to provide energy to the interface between aptamers and the sample fluid is implanted into the skin along with the aptamers. In various embodiments, the device may use mechanical energy, acoustic energy, or the energy may be an electrical field.

### EXAMPLES

### Example 1

In one embodiment, a wearable device such as 100 or 102 has a thickness of its housing 110 or 112 that is less than 5 mm and ideally less than 3 mm thick, similar to Abbott's Libre 2 and Libre 3 continuous glucose monitor wearables. While electrochemical sensors may easily have a sensor element 120 or 190 that used an electrode that can connect to the surface of a circuit board or which can be bent at 90 degrees, optical waveguides cannot redirect light over tight bends or curvatures without significant if not total loss of optical signal (due to exceeding the optical critical angle for total internal reflection). FIG. 10 illustrates at least one example where a mirror, beam splitter, or other optical component is able to couple light to at least one optical detector and at least one optical source, such that the device housing 1010 (or other device housings in other figures) can be at least less than 5 mm thick. An optical fiber that is 500 μm in diameter would require a beam splitter or 45 degree mirror that is less than 1 mm thick, which with respect to the horizontal surface of skin could re-orient dominantly vertically traveling light from a waveguide sensor into a predominately horizontal direction which it is coupled to opto-electronic components (detectors, sources) mounted on a circuit board.

### Example 2

One of the most potentially beneficial features of the embodiments of the present invention is the ability to provide strong signaling even with larger sized analytes. This advantage exists because a longer nucleotide base with more binding sites to the larger analyte can be used, because for an aptamer on a waveguide with fluorescent tagging, the fluorescent probe distance is less sensitive to distance than for example and electrochemical format. A first example that may be used with embodiments of the present invention is sensing of NT-proBNP using the sequence:

SEQ ID NO: 1:
GGCAGGAAGACAAACAGGTCGTAGTGGAAACTGTCCACCGTAGACC
GGTTATCTAGTGGTCTGTGGTGCTGT

or using BNP aptamers from the literature or using NT-proBNP or BNP aptamers from commercial sources such as SomaLogic who have developed aptamers for capture of >10,000 peptides and proteins. The binding portion of an aptamer sequence can form a stem-loop configuration and has, as a non-limiting example, multiple thymine locations that can be optically tagged such as a fluorescent tag and a quencher that are brought close in the absence of the analyte:

SEQ ID NO: 2:
CACCGTAGACCGGTTATCTAGTGGTCTGTGGTG

Alternately the one or more sequences can be tagged such that two or more optical tags are brought in close proximity for quenching or fluorescence resonance transfer when binding to NT-proBNP. A second example that may be used with embodiments of the present invention is sensing of monomeric c-reactive protein, for example using the sequence:

SEQ ID NO: 3:
ACA CGATGG GGG GGTATG ATT TGA TGT GGT TGT TGC
ATG ATC GTG G

which forms a stem geometry near its 3′ and 5′ ends which can be exploited for tagging, and extended further in stem length with additional base pairs if needed. A third example that may be used with embodiments of the present invention is sensing of pentameric c-reactive protein, for example using the sequence 5′-SEQ. ID NO: 4-3′, where:

SEQ. ID NO: 4 is:
GCCTGTAAGGTGGTCGGTGTGGCGAGTGTGTTAGGAGAGATTGC

and for which stems form and can be optimized and tagged near the 5′ end of the aptamer. A fourth example would be chemically modified aptamers, such as Somalogic SOMAMERs SL1025 and SL1026 for the inflammatory analyte IL-6 which are commercially available and can be provided by SomaLogic with specific designs provided to them using embodiments of the present invention including tagging and substrate bonding to glasses, with aptamer details as taught in the literature such as: Hirota M, Murakami I, Ishikawa Y, Suzuki T, Sumida S, Ibaragi S, Kasai H, Horai N, Drolet D W, Gupta S, Janjic N, Schneider D J. Chemically Modified Interleukin-6 Aptamer Inhibits Development of Collagen-Induced Arthritis in Cynomolgus Monkeys. Nucleic Acid Ther. 2016 February; 26 (1): 10-9. doi: 10.1089/nat.2015.0567. Epub 2015 Nov. 18. PMID: 26579954; PMCID: PMC4753578. A fifth example that may be used with embodiments of the present invention is sensing of BNP, for example using

SEQ. ID NO: 5:
TTT TTT GGC GAT TCG TGA TCT CTG CTC TCG GTT
TCG CGT TCG TTC

and which has a stem region near its end regions and multiple thymine attachment points for optical tags near the stem region. A sixth example that may be used with embodiments of the present invention is sensing of Troponin, for example using SEQ. ID NO: 6: TTT TTT CGT GCA GTA CGC CAA CCT TTC TCA TGC GCT GCC CCT and which has a stem region near with two thymine attachment points for optical tags near the stem region.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for continually sensing at least one analyte in a sample fluid via measurement of the analyte, comprising:
at least one optically transparent component adapted for in-vivo placement;
a plurality of aptamers bound to the optically transparent component wherein the aptamers are capable of binding to the analyte and wherein the aptamers carry at least one tag that changes in at least one optically measurable property when the aptamers bind to the analyte; and
at least one optical source and detector coupled to the optically transparent component capable of measuring the optically measurable property of the aptamers.

2. The device of claim 1 wherein the aptamers comprise at least one fluorescent tag, and at least one optical quencher tag.

3. The device of claim 1 wherein the aptamers comprise at least one first fluorescent tag, and at least one second fluorescent tag, wherein energy is transferred from the first fluorescent tag to the second fluorescent tag.

4. The device of claim 1 wherein the optically transparent component is porous and carries aptamers internally.

5. The device of claim 1 wherein the optically transparent component is coated with a porous material that carries aptamers.

6. The device of claim 1 comprising pores that have an average size selected from the group consisting of greater than 3, 10, and 30 nm and a width selected from the group consisting of less than 50, 100, 200, and 500 nm.

7. The device of claim 1 wherein the optically transparent material is a waveguide.

8. The device of claim 4 wherein the optically transparent component is a hydrogel.

9. The device of claim 4 wherein the optically transparent component is porous silica.

10. The device of claim 1 wherein the plurality of aptamers includes at least a first subset of aptamers that bind to a first analyte and a second subset of aptamers that bind to a second analyte.

11. The device of claim 1 wherein the optically transparent material is a plurality of waveguides.

12. The device of claim 1 wherein at least 90% of the aptamers will remain attached for at least 3 days when tested for 3 days of operation in serum at 37 degrees Celsius.

13. The device of claim 1 wherein the porous material has a porous surface area of at least 20 m²/g.

14. The device of claim 1 wherein the optically transparent component has a surface area to volume that is greater than 40,0000 by an amount selected from the group consisting of 2×, 10×, 50×, 200×, and 1000× greater than the equivalent surface area to volume of a smooth planar 100 μm diameter waveguide.

15. The device of claim 1 wherein the optically transparent component is a bundle of optical fibers with at least 1 micrometer of separation between the optical fibers.

16. The device of claim 1 wherein the optically transparent component is non-cylindrical.

17. The device of claim 1 wherein the aptamers comprise a plurality of fluorescent tags that are separated by at least 5 nm.

18. The device of claim 1 wherein the optically transparent component has a surface and the surface comprises the aptamers and an antifouling chemistry.

19. The device of claim 18 wherein the antifouling chemistry has a mixed charge at the interface with the sample fluid.

20. The device of claim 18 wherein the antifouling chemistry has zwitterionic charge.

21. The device of claim 18 wherein the antifouling chemistry has a charge.

22. The device of claim 18 wherein the antifouling chemistry has a net charge and the charge is negative.

23. The device of claim 21 wherein the charge is less than 50% of an equivalent of a net charge of 1 charge per nm².

24. The device of claim 18 wherein the antifouling chemistry has a plurality of charged molecular brushes interspersed in between the aptamers.

25. The device of claim 24 wherein the molecular brushes are >2 nm in length.

26. The device of claim 24 further comprising a protective layer that protects the surface from fouling in between the aptamers, wherein the protective layer comprises a monolayer of mixed charge molecules that form a boundary with the sample fluid, the boundary layer and the mixed charge monolayer having a Debye length, and further, wherein the molecular brush molecules have a net charge near the aptamers which extend the Debye length from the boundary layer with the mixed charge monolayer by at least twice the length.

27. The device of claim 1 further comprising at least one membrane between the optically transparent component and the sample fluid.

28. The device of claim 1 further comprising at least one mechanically protective element between the optically transparent component and the sample fluid.

29. The device of claim 28 wherein the mechanically protective element is porous to the analyte.

30. The device of claim 1 wherein the optically transparent component is formed on at least one metal wire.

31. The device of claim 30 wherein the metal wire is tantalum.

32. The device of claim 1 wherein the optically transparent component has a tensile strength of >50 mPa.

33. The device of claim 1 wherein the optically transparent component has a tensile strength of >100 mPa.

34. The device of claim 1 wherein the optically transparent component comprises polyimide and the tag is selected from the group consisting of red fluorescent tags and near-infrared fluorescent tags.

35. The device of claim 1 wherein the optically transparent component is a waveguide sensor formed of at least one polymer and at least one glass, where the glass is the waveguide material and the polymer is at least one material capable of preventing breakage and material loss of the device.

36. The device of claim 1 wherein the optically transparent component is a waveguide sensor with an aspect ratio that is less than 10:1.

37. The device of claim 1 wherein the optically transparent component comprises at least one insertion material and at least one waveguide sensor such that the waveguide sensor is placed entirely in either a user's dermis or hypodermis.

38. The device of claim 1 wherein the optically transparent component is comprised of a plurality of waveguides and insertion points into the skin that share at least one common optical emitter or detector.

39. The device of claim 1 wherein the optically transparent component is one or more waveguide sensors located inside the lumen of a hollow needle placed into a user's skin.

40. The device of claim 1 wherein the optically transparent component is least one microneedle sensor that less than 1 mm in length in insertion into a user's skin and which has a waveguide aspect ratio of >10:1.

41. The device of claim 1 wherein the optically transparent component is one microneedle element that penetrates skin by <1 mm and which rejects a percentage of autofluorescence from skin selected from the group consisting of 30, 60, 90, and 95%.

42. The device of claim 1 wherein the analyte is NT-proBNP.

43. The device of claim 1 wherein the analyte is pentameric C-reactive protein.

44. The device of claim 1 wherein the analyte is monomeric C-reactive protein.

45. The device of claim 1 wherein the analyte is IL-6.

46. The device of claim 1 wherein the analyte is BNP.

47. The device of claim 1 wherein the analyte is Troponin.

48. The device of claim 1 wherein the optically transparent component is a planar waveguide.

49. The device of claim 48 further comprising a substrate, wherein the substrate is not a waveguide, and the substrate comprises an optical cladding that carries the planar waveguide.

50. The device of claim 1 wherein the at least one optically measurable property is fluorescence intensity.

51. The device of claim 1 wherein the at least one optically measurable property is the ratio between two fluorescence intensities from two tags.

52. The device of claim 1 wherein the at least one optically measurable property is fluorescence wavelength.

53. The device of claim 1 wherein the at least one optically measurable property is fluorescence lifetime.

54. The device of claim 1 wherein the at least one optically measurable property has less than 30% signal loss over at least one day, or at least one week of continuous in-vivo operation.

55. The device of claim 1 wherein the device further comprises a mechanical housing and the mechanical housing is less than 5 mm thick.

56. The device of claim 1 wherein the optical tag further comprises at least one photostabilizer molecule.

57. The device of claim 1 wherein the device is capable of being applied to a user's skin and the sample fluid is interstitial fluid.

58. The device of claim 1 further comprising at least one element to provide energy to the interface between aptamers and the sample fluid, to remove non-specific binding solutes from the aptamers.

59. The device of claim 58 wherein the device is capable of being applied to a user's skin and the sample fluid is interstitial fluid, and further, wherein the at least one element to provide energy to the interface between aptamers and the sample fluid is located outside the skin.

60. The device of claim 58 wherein the device is capable of being applied to a user's skin and the sample fluid is interstitial fluid, and wherein the at least one element to provide energy to the interface between aptamers and the sample fluid is implanted into the skin along with the aptamers.

61. The device of claim 58 where the energy is mechanical.

62. The device of claim 58 where the energy is acoustic.

63. The device of claim 58 where the energy is electrical field generated by an alternating charge of magnitude by a value that is selected from the group consisting of 1, 10, and 100 nC/cm².

64. A method for continually sensing at least one analyte in a sample fluid via measurement of the analyte, comprising:
a. at least one optically transparent component adapted for in-vivo placement;
b. a plurality of aptamers bound to the optically transparent component wherein the aptamers bind to the analyte and carry at least one tag that changes in at least one optically measurable property when the aptamers bind to the analyte;
c. at least one optical source and detector coupled to the optically transparent component capable of measuring the optically measurable property of the aptamers;
d. an optical source for exciting fluorescence of the at least one tag;
wherein the optical source is repeatedly but not constantly introduced to the optically transparent material with a duty cycle; and further, wherein the duty cycle is a percentage of time during which the sensor is placed in the sample fluid, wherein the percentage of time is selected from the group consisting of less than 20%, 5%, 1%, 0.2%, 0.05%, and 0.01%.
